(19) United States
(12) Patent Application Publication
(10) Pub. No.: US 2023/0125697 A1
(43) Pub. Date: Apr. 27, 2023

(54) CANNABINOID COMPOSITIONS AND AEROSOLS

(71) Applicant: ROXX LABS LTD., Migdal Haemeq (IL)

(72) Inventor: Miron HAZANI, Haifa (IL)

(21) Appl. No.: 17/911,301

(22) PCT Filed: Mar. 21, 2021
(86) PCT No.: PCT/IL2021/050311

§ 371 Date: Sep. 13, 2022

(30) Foreign Application Priority Data
Mar. 24, 2020 (IL) 273561

(51) Int. Cl. A61K 31/352; A61K 31/05; A61K 36/185; A61K 9/00; A24B 15/167

(52) U.S. Cl. CPC A61K 31/352; A61K 31/05; A61K 36/185; A61K 9/007; A24B 15/167; A61K 2236/00

(57) ABSTRACT

The present disclosure generally relates to aqueous compositions comprising a cannabinoid acid or a salt thereof, processes for the preparation thereof and uses thereof for inhalation.

### TECHNICAL FIELD

The present disclosure generally relates to aqueous compositions comprising a cannabinoid acid or a salt thereof, processes for the preparation thereof and uses thereof for inhalation.

### BACKGROUND

Cannabinoids are a diverse class of chemical compounds that act as ligands to the cannabinoid receptors. The clinical usefulness of the cannabinoids, including Δ⁹-tetrahydrocannabinol (Δ⁹-THC), to provide analgesia, help alleviate nausea and emesis, as well as stimulate appetite has been well-recognized. Cannabinoids offer a variety of pharmacological benefits, including, but not limited to, anti-spasmodic, anti-inflammatory, anti-convulsant, anti-oxidant, neuroprotective, reducing pain, anti-cancer, and immunomodulatory effects.

The principle cannabinoids present in herbal cannabis are cannabinoid acids Δ⁹-tetrahydrocannabinolic acid (Δ⁹-THCA) and cannabidiolic acid (CBDA) with small amounts of the respective neutral (decarboxylated) cannabinoids—tetrahydrocannabinol and cannabidiol (CBD). In addition, cannabis may contain lower levels of other minor cannabinoids. The relative levels of THCA, CBDA, THC and CBD in the plant material typically depend on the plant species, with specific species being engineered to contain high level of the THC derivatives (THC and THCA)

Tetrahydrocannabinolic acid (THCA) is a non-psychoactive natural precursor of tetrahydrocannabinol (THC). THCA is found in variable quantities in fresh, undried cannabis, but is progressively decarboxylated to THC with drying, and especially under intense heating such as when cannabis is smoked or cooked into cannabis edibles.

THC is highly insoluble in water and is typically used in oily compositions. THCA is more soluble in water. However, typical THC compositions are provided as viscous suspensions and/or in organic solvents, such as vegetable glycerin (VG) or propylene glycol (PG).

U.S. Pat. No. 7,524,881 is directed to isolation and purification of THC. It disclosed the extraction of cannabis plant material with heptane, basic extraction of the organic phase with sodium hydroxide at a pH of 12.7-13.2, further extraction of the resultant basic aqueous phase with isopropyl ether, acidification, and treatments with florisil and charcoal to form THC solution, which is then concentrated and purified.

Hazekamp et al. (Preparative Isolation of Cannabinoids from Cannabis sativa by Centrifugal Partition Chromatography, Journal of Liquid Chromatography & Related Technologies 27(15):2421-2439 December 2004) discloses a method is presented for the preparative isolation of seven major cannabinoids from Cannabis sativa plant material. Hazekamp discloses the use of a two solvent system, to obtain purified samples of the cannabinoids; (−)-Δ⁹-(trans)-tetrahydrocannabinol (Δ⁹-THC), cannabidiol (CBD), cannabinol (CBN), cannabigerol (CBG), (−)-Δ⁹-(trans)-tetrahydrocannabinolic acid-A (THCA), cannabigerolic acid (CBGA) and cannabidiolic acid (CBDA).

Stepanov I and Naomi F (“Bringing attention to e-cigarette pH as an important element for research and regulation”, Tobacco Control 24(4), p. 413-414) discusses pH effects on nicotine in electronic cigarettes.

US 2014/166028 discloses a method for the administration of nicotine, THC, tobacco, cannabidiol or a base alkaloid. The method includes administering in the oral or nasal cavity an absorption conditioning unit having at least two agents selected from the group consisting of (a) a buffer agent, (b) a capturing agent, (c) a penetration agent, and (d) a thermal agent, to the mammal, and then administering by inhalation a bioactive agent selected from the group consisting of nicotine, THC, cannabidiol and a base alkaloid. The absorption conditioning unit may be in a dosage form not containing a drug. The absorption conditioning unit may create a pH in the oral cavity or nasal cavity of 7.8-10 for a period of ten minutes or more after administration, the dosage form not containing an acid and not containing a drug.

US 2014/209109 discloses a pharmaceutical composition and administration apparatus including a portable powered vaporizer with a mouthpiece, smokeless vaporizing element, and a removable chamber. The chamber contains a composition comprising tetrahydrocannabinol (THC), cannabidiol (CBD), FCC grade ethanol, flavoring, pharmaceutical grade nicotine, USP grade aqueous glycerin, USP/EP grade propylene glycol, and USP grade vegetable glycerin.

WO 2016/019353 discloses a pharmaceutical formulation of cannabis compounds suitable for pulmonary delivery to a patient, comprising: a volatile liquid, wherein the volatile liquid comprises a mixture of: a cannabis oil extract having a known amount of a medicinal cannabis compound; and an aerosol precursor. The volatile liquid may be in an inhalable form, in an aerosol form and/or in vapor form.

There is an unmet need for cannabinoid compositions suitable for the generation of an inhalable aerosol, which are water-based devoid of organic solvents.

### SUMMARY

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present invention provides aqueous solutions of cannabinoids which are suitable and useful for inhalation by a subject. These aqueous solutions are achieved by forming salts of the cannabinoid acids found in the cannabis plant by contact with an aqueous base, at a pH of 9 or higher. Advantageously the cannabinoid salts so formed are stable in the aqueous solution. Furthermore, the cannabinoid salts can be generated without extraction of the cannabinoids by use of organic solvents or organic co-solvents.

Provided herein are cannabinoid compositions suitable for the administration of a cannabinoid via inhalation, according to some embodiments. According to some embodiments, the compositions are for use in the administration via inhalation. Advantageously, the cannabinoid compositions disclosed herein are provided in an aqueous medium, such as an aqueous medium, which is substantially devoid of organic solvents, according to some embodiments. According to some embodiments, the cannabinoid composition comprises an aqueous solution comprising at least one cannabinoid compound, such as a cannabinoid acid or a salt thereof. According to some embodiments, the aqueous solution has a pH of at least 8.5. According to some embodiments, the aqueous solution has a pH of at least 9.

According to some embodiments, there is provided a cannabinoid composition suitable for administration of a cannabinoid via inhalation, the composition comprises an aqueous solution comprising at least one cannabinoid compound, wherein the aqueous solution has a pH of at least 8.5.

According to some embodiments, the administration of the cannabinoid via inhalation comprises generating an inhalable aerosol upon heating the cannabinoid composition in an electronic vaporizer. According to some embodiments, the electronic vaporizer is an electronic vaping device or an electronic cigarette.

According to some embodiments, the inhalable aerosol has a pH of at least 9. According to some embodiments, the inhalable aerosol has a pH in the range of 5.5 to 7.5.

According to some embodiments, the aqueous solution has a pH of at least 10. According to some embodiments, aqueous solution has a pH in the range of 10.5 to 11.5.

According to some embodiments, the concentration of the at least one cannabinoid compound in the aqueous solution is in the range of 0.1% to 10% w/w. According to some embodiments, the concentration of the at least one cannabinoid compound in the aqueous solution is in the range of 1% to 10% w/w. According to some embodiments, the concentration of the at least one cannabinoid compound in the aqueous solution is in the range of 2% to 10% w/w. According to some embodiments, the concentration of the at least one cannabinoid compound in the aqueous solution is in the range of 4% to 6% w/w.

According to some embodiments, the at least one cannabinoid compound is extracted from a plant material, wherein the plant is of cannabis genus.

According to some embodiments, the at least one cannabinoid compound comprises tetrahydrocannabinol (THC), tetrahydrocannabinolic acid (THCA), cannabidiol (CBD), cannabidiolic acid (CBDA), salts thereof or a combination thereof. According to some embodiments, the at least one cannabinoid compound comprises THCA or a salt thereof.

According to some embodiments, the cannabinoid composition further comprises at least one additive selected from the group consisting of a propellant, an anti-coughing agent and a flavorant.

According to some embodiments, the cannabinoid composition is substantially devoid of organic solvents. According to some embodiments, the cannabinoid composition less than 10% w/w organic solvents. According to some embodiments, the cannabinoid composition less than 2% w/w organic solvents.

According to some embodiments, the cannabinoid composition is in liquid form and comprising at least 90% w/w water.

According to some embodiments, the cannabinoid composition is for use in the administration of the cannabinoid via inhalation.

According to some embodiments, there is provided a vaporization filling composition comprising the cannabinoid composition of the present invention. According to some embodiments, there is provided a vaporization filling composition consisting essentially of the cannabinoid composition of the present invention. According to some embodiments, there is provided a vaporization filling composition consisting of the cannabinoid composition of the present invention.

According to some embodiments, the vaporization filling composition is selected from an electronic cigarette cartridge filling composition and a vaporizer cartridge filling composition. According to some embodiments, the vaporization filling composition is an electronic cigarette cartridge filling composition. According to some embodiments, the vaporization filling composition is a vaporizer cartridge filling composition. According to some embodiments, the vaporization filling composition is a vaping device cartridge filling composition.

According to some embodiments, the aqueous solution is prepared by a process comprising the steps of:
(a) contacting cannabis plant material with an aqueous base, to form an aqueous solution comprising the at least one cannabinoid, and a water insoluble plant material; and
(b) separating the aqueous solution comprising the at least one cannabinoid from the insoluble plant material.

According to some embodiments, the aqueous base comprises sodium hydroxide, potassium hydroxide, lithium hydroxide, magnesium hydroxide, sodium carbonate, potassium carbonate or a combination thereof. According to some embodiments, the aqueous base is selected from the group consisting of aqueous sodium hydroxide, aqueous potassium hydroxide, aqueous lithium hydroxide, aqueous magnesium hydroxide, aqueous sodium carbonate and aqueous potassium.

According to some embodiments, the aqueous base comprises a hydroxide anion at a concentration in the range of 0.001M to 0.5M. According to some embodiments, the aqueous base comprises a hydroxide anion at a concentration in the range of 0.1M to 0.5M. According to some embodiments, the aqueous base comprises a hydroxide anion at a concentration in the range of 0.001M to 0.5M.

According to some embodiments, the process further comprises a step of grinding the cannabis plant material prior to step (a). According to some embodiments, the contacting of step (a) is maintained for at least 1 hour. According to some embodiments, the separation of step (b) is performed by centrifugation.

According to some embodiments, step (a) further comprises macerating the cannabis plant material in the aqueous base.

According to some embodiments, the cannabis plant material comprises tetrahydrocannabinolic acid (THCA).

According to some embodiments, the cannabis plant material comprises a THCA-enriched cannabis species.

According to some embodiments, the process is devoid of steps of extraction with an organic solvent.

According to some embodiments, the process further comprises the steps of:
(c) adding an acid to the aqueous solution comprising the at least one cannabinoid to a pH in the range of 1-5, thereby precipitating the at least one cannabinoid and forming an acidic aqueous solution;
(d) separating the precipitated at least one cannabinoid from the acidic aqueous solution; and
(e) dissolving the precipitated at least one cannabinoid in a second aqueous base, thereby forming a purified aqueous solution comprising the at least one cannabinoid.

According to some embodiments, the acid is a mineral acid. According to some embodiments, the pH of the acidic aqueous solution of step (c) is in the range of 3.5 to 4.5.

According to some embodiments, the separation of step (d) is performed by centrifugation.

According to some embodiments, the second aqueous base of step (e) comprises a hydroxide anion at a concentration in the range of 0.001M to 0.5M. According to some embodiments, the second aqueous base of step (e) comprises a hydroxide anion at a concentration in the range of 0.1M to 0.5M.

According to some embodiments, there is provided a method of delivering a cannabinoid to a user of an electronic vaporizer via inhalation, the method comprising the steps of:
(i) providing a cannabinoid composition comprising an aqueous solution comprising at least one cannabinoid compound, wherein the aqueous solution has a pH of at least 8.5; and
(ii) aerosolizing the cannabinoid composition of step (a) with an electronic vaporizer, to form an inhalable aerosol

According to some embodiments, the inhalable aerosol is inhaled by the user of the electronic vaporizer.

According to some embodiments, the aqueous solution of step (i) has a pH of at least 9. According to some embodiments, inhalable aerosol formed in step (ii) has a pH in the range of 5.5 to 7.5.

According to some embodiments, the aerosolizing of step (ii) comprises heating the cannabinoid composition of step (i) with the electronic vaporizer.

According to some embodiments, the aqueous solution of step (i) has a pH in the range of 10.5 to 11.5.

According to some embodiments, the concentration of the at least one cannabinoid compound in the aqueous solution of step (i) is in the range of 4% to 6% w/w.

According to some embodiments, the at least one cannabinoid compound is extracted from a plant material, wherein the plant is of cannabis genus.

According to some embodiments, the at least one cannabinoid compound comprises tetrahydrocannabinol (THC), tetrahydrocannabinolic acid (THCA), cannabidiol (CBD), cannabidiolic acid (CBDA), salts thereof or a combination thereof. According to some embodiments, the at least one cannabinoid compound comprises THCA or a salt thereof.

According to some embodiments, the cannabinoid composition of step (i) is substantially devoid of organic solvents.

According to some embodiments, the cannabinoid composition of step (i) is in liquid form and comprising at least 90% w/w water.

According to some embodiments, the aqueous solution of step (i) is prepared by a process comprising the steps of:
(a) contacting cannabis plant material with an aqueous base, to form an aqueous solution comprising the at least one cannabinoid, and a water insoluble plant material; and
(b) separating the aqueous solution comprising the at least one cannabinoid from the insoluble plant material.

According to some embodiments, step (a) further comprises macerating the cannabis plant material in the aqueous base. According to some embodiments, the process of preparing aqueous solution of step (i) is devoid of steps of extraction with an organic solvent.

According to some embodiments, the process of preparing aqueous solution of step (i) further comprises the steps of:
(c) adding an acid to the aqueous solution comprising the at least one cannabinoid to a pH in the range of 1-5, thereby precipitating the at least one cannabinoid and forming an acidic aqueous solution;
(d) separating the precipitated at least one cannabinoid from the acidic aqueous solution; and
(e) dissolving the precipitated at least one cannabinoid in a second aqueous base, thereby forming a purified aqueous solution comprising the at least one cannabinoid.

According to some embodiments, there is provided an electronic vaporizer cartridge comprising a liquid container, wherein the liquid container contains a cannabinoid composition comprising an aqueous solution comprising at least one cannabinoid compound, wherein the aqueous solution has a pH of at least 8.5.

According to some embodiments, the aqueous solution has a pH of at least 9. According to some embodiments, the aqueous solution has a pH in the range of 10.5 to 11.5. According to some embodiments, concentration of the at least one cannabinoid compound in the aqueous solution is in the range of 4% to 6% w/w.

According to some embodiments, the at least one cannabinoid compound comprises THCA or a salt thereof.

According to some embodiments, the cannabinoid composition is substantially devoid of organic solvents. According to some embodiments, the cannabinoid composition is in liquid form and comprises at least 90% w/w water.

According to some embodiments, the aqueous solution is prepared by a process comprising the steps of:
(a) contacting cannabis plant material with an aqueous base, to form a suspension comprising an aqueous solution, which comprises the at least one cannabinoid, and a water insoluble plant material; and
(b) separating the aqueous solution comprising the at least one cannabinoid from the insoluble plant material.

According to some embodiments, the aqueous solution is prepared by a process comprising the steps of:
(a) contacting cannabis plant material with an aqueous base, to form an aqueous solution comprising the at least one cannabinoid, and a water insoluble plant material; and
(b) separating the aqueous solution comprising the at least one cannabinoid from the insoluble plant material.

According to some embodiments, there is provided an aerosol composition comprising tetrahydrocannabinol (THC) at a total weight of 1-8% w/w based on the total weight of the aerosol composition, and water 70-99% w/w based on the total weight of the aerosol composition, wherein the aerosol comprising droplets having an mass median aerodynamic diameter (MMAD) of at most 5 microns. According to some embodiments, the aerosol composition further comprises tetrahydrocannabinolic acid (THCA).

According to some embodiments, the aerosol composition is having a pH in the range of 5.5 to 7.5.

According to some embodiments, the aerosol composition comprises droplets having a mass median aerodynamic diameter (MMAD) of at most 5 microns. According to some embodiments, the aerosol composition comprises droplets having a mass median aerodynamic diameter (MMAD) of at most 10 microns.

According to some embodiments, the aerosol composition is prepared by aerosolizing a cannabinoid composition comprising an aqueous solution comprising THCA, wherein the aqueous solution has a pH of at least 8.5. According to some embodiments, the aqueous solution has a pH of at least 9.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

FIG. 1 shows two overlaying HPLC chromatograms; a chromatogram of the aqueous formulation disclosed herein (dotted line); and a chromatogram of an elution of an aerosol produced from aerosolizing the aqueous formulation disclosed herein (full line).

FIG. 2 is a chart representing Mass Distribution on Impactor parts in an aerosol depicting the relative mass of an aerosol produced from aerosolizing the aqueous formulation disclosed herein, in each particle diameter size group.

FIG. 3 shows Mass Distribution on Impactor parts in an aerosol produced from aerosolizing the aqueous formulation disclosed herein.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Provided herein are cannabinoid compositions suitable for the administration of a cannabinoid via inhalation, according to some embodiments. Advantageously, the cannabinoid compositions disclosed herein are provided in an aqueous medium, such as an aqueous medium, which is substantially devoid of organic solvents, according to some embodiments. In addition, the THC compositions disclosed herein, when delivered via inhalation, exhibit THC delivery properties similar to cigarettes, thereby providing an efficient substitute for smokers, which is safer and is devoid of smoke harmful organic decomposition contaminants. According to some embodiments, the cannabinoid composition comprises an aqueous solution comprising at least one cannabinoid compound, such as a cannabinoid acid or a salt thereof. According to some embodiments, the aqueous solution has a pH of at least 8.5. According to some embodiments, the aqueous solution has a pH of at least 9. According to some embodiments, the cannabinoid composition consists of an aqueous solution comprising at least one cannabinoid compound, such as a cannabinoid acid or a salt thereof. According to some embodiments, the aqueous solution has a pH of at least 8.5 or at least 9.

The term “solution” as used herein broadly refers to a combination, mixture and/or admixture of ingredients having at least one liquid component. Thus, the term “aqueous solution” refers to any solution, in which at least one of its liquid components is water, wherein at least 50% of its weight is water. Aqueous solutions typically include water in greater quantity or volume than a solute. Typical additional solvents include alcohols, aldehydes, ketones, sulfoxides, sulfones, nitriles and/or any other suitable solubilizing molecule or carrier compound. Preferably, “solution” refers broadly to a mixture of miscible substances, where one substance dissolves in a second substance. More preferably, in a solution the essential components are homogeneously mixed and that the components are subdivided to such an extent that there is no appearance of light scattering visible to the naked eye when a one inch diameter bottle of the mixture is viewed in sunlight.

According to some embodiments, there is provided a cannabinoid composition suitable for use in the administration of a cannabinoid via inhalation, the composition comprises an aqueous solution comprising at least one cannabinoid compound, wherein the aqueous solution has a pH of at least 8.5.

The phrases “suitable for use in the administration via inhalation”, “suitable for administration via inhalation” and “suitable for inhalation” are interchangeable and refer to compositions, that may be administered to a human subject without the subject experiencing undue toxicity and the like, and effective for the intended use of the administration (e.g. therapeutic or recreational). For example, several extraction processes of cannabis products employ organic solvents and/or other chemical, which are hazardous to the human health and are present in the extracted composition. Such not purified compositions are examples of compositions, which are not suitable for administration via inhalation. According to some embodiments, the cannabinoid composition is formulated for inhalation.

According to some embodiments, there is provided a cannabinoid composition for use in the administration of a cannabinoid via inhalation, the composition comprises an aqueous solution comprising at least one cannabinoid compound, wherein the aqueous solution has a pH of at least 8.5.

According to some embodiments, the composition is provided in a kit, wherein the kit comprises the cannabinoid composition of the present invention and further comprises instructions for use via inhalation.

According to some embodiments, there is provided a cannabinoid composition, the composition comprises an aqueous solution comprising at least one cannabinoid compound, wherein the aqueous solution has a pH of at least 8.5. According to some embodiments, there is provided a cannabinoid composition, the composition comprises an aqueous solution comprising at least one cannabinoid compound, wherein the aqueous solution has a pH of at least 8.5, wherein the cannabinoid composition is suitable for administration via inhalation.

According to some embodiments, the cannabinoid composition is consisting of the aqueous solution.

The term “cannabinoid”, as used herein, includes all major and minor cannnabinoids found in natural cannabis and hemp material that can be isolated from a natural source or reproduced by synthetic means. This includes delta-9-Tetrahydrocannabinol (THC), delta-9-Tetrahydrocannabinolic acid (THCA), delta-8-Tetrahydrocannabinol, Cannabidiol (CBD), Cannabidiolic acid (CBDA), Cannabinol (CBN), Cannabinolic acid (CBNA), tetrahydrocannabinovarin (THCV), cannabidivarin (CBDV), cannabigerol (CBG), cannabigerolic acid (CBGA) and cannabichromene (CBC). The term “cannabinoid” also includes basic salts of the acid mentioned above, for example, THCA-sodium salt and THCA-potassium salt.

The term “tetrahydrocannabinolic acid” and “THAC acid” are interchangeable and refer to common derivatives of THC, which are substituted in position 2 of the aromatic ring by a carboxylic acid. THC has two dominant isomers, Δ⁹-THC and Δ⁸-THC. Accordingly, THCA has corresponding Δ⁹ and Δ⁸ isomers. The chemical structures of the parent tetrahydrocannabinols (Δ⁹-THC and Δ⁸-THC) and tetrahydrocannabinolic acids (Δ⁹-THCA and Δ⁸-THCA) are presented below:

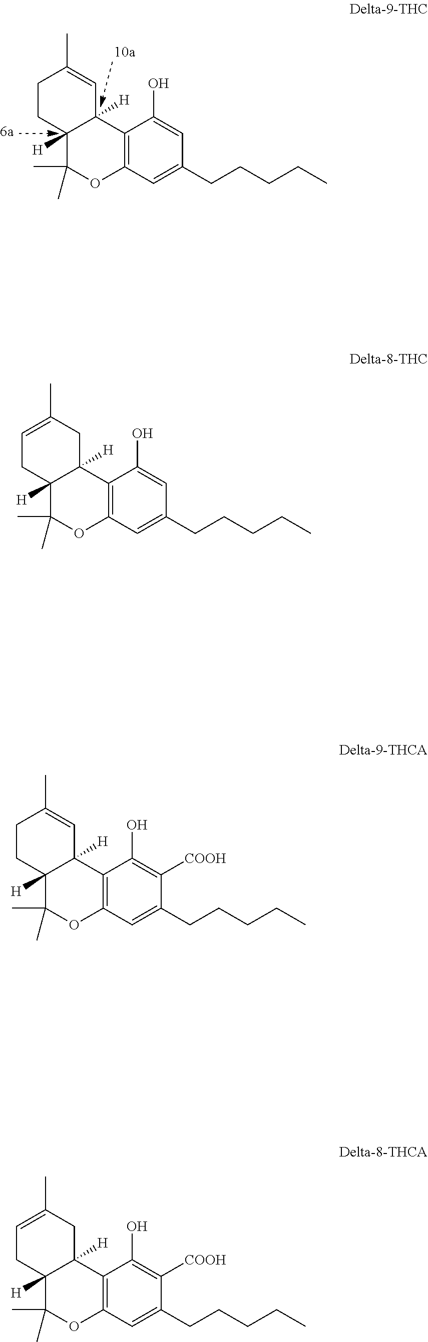

It is to be understood that although the natural THC isomers include an n-C₅H₁₁ chain in position 3, derivatives of THC may include other substituents. Therefore, the term tetrahydrocannabinolic acid includes corresponding structures, in which position 3 is substituted by a group, which is either an n-C₅H₁₁ or a different chemical group.

The term “tetrahydrocannabinolic acid” should be interpreted broadly referring to all possible stereoconfigurations and salts of the relevant formula. Specifically, the natural THC includes two vicinal asymmetric positions, position 6a and position 10a, as shown above. The two vicinal asymmetric positions exist in trans relative configuration, and both are designated R absolute configuration. Thus, the (6aR,10a1?) absolute configuration is the preferred configuration for tetrahydrocannabinolic acids of the current invention, however, said tetrahydrocannabinolic acids are not limited to this configuration According to some embodiments, positions 6a and 10a of the THCA acid are in trans relative configuration. According to some embodiments, position 6a has R absolute configuration. According to some embodiments, position 10a has R absolute configuration. According to some embodiments, the THCA acid has the (6aR,10aR) absolute configuration.

As used herein the terms “formulation” and “compositions” generally refer to any mixture, solution, suspension or the like that contains an active ingredient, such as cannabinoid, and, optionally, a carrier. The carrier may be any carrier acceptable for smoking, that is compatible for delivery with the active agent.

According to some embodiments, the administration of the cannabinoid via inhalation comprises generating an inhalable aerosol of the cannabinoid composition. According to some embodiments, the administration of the cannabinoid via inhalation comprises generating an inhalable aerosol of the cannabinoid composition upon heating the cannabinoid composition in an aerosol generating device. According to some embodiments, the administration of the cannabinoid via inhalation comprises generating an inhalable aerosol of the cannabinoid composition upon heating the cannabinoid composition in an electronic vaporizer. According to some embodiments, the administration of the cannabinoid via inhalation comprises generating an inhalable aerosol upon heating the cannabinoid composition in an electronic vaporizer.

Surprisingly, it was found that the basic (pH of at least 8.5 or at least 9, or at least 10) is suitable for delivery to electronic vaporizer users. Specifically, although such basic compositions are not suitable for direct use of human, it was found that upon aerosolization, a substantially pH neutral aerosol formed, which is compatible with inhalation. Without wishing to be bound by any theory of mechanism of action, the basic cannabinoid composition comprises non-volatile bases, which are not aerosolized, and organic material, comprising THCA, present mainly as a basic salt, e.g. THCA-sodium salt. Upon heating and aerosolization with the electronic vaporizer, THCA-sodium salt, which is in equilibrium with THCA, undergoes decarboxylation to form THC, which is aerosolized together with the water medium. THC is pH neutral, therefore, the aerosol is substantially neutral and suitable for the use of human subjects.

According to some embodiments, the inhalable aerosol has a pH in the range of 5.5 to 8.5. According to some embodiments, the inhalable aerosol has a pH in the range of 6.0 to 7.5. According to some embodiments, the inhalable aerosol has a pH in the range of 6.5 to 7.5.

Another requirement from the generated aerosol is to have droplets having the required size so as to reach the lungs of the electronic vaporizer user. These requirements are detailed below. According to some embodiments, the aerosol has droplets having an mass median aerodynamic diameter (MMAD) of at most 5 microns.

According to some embodiments, there is provided a cannabinoid composition for use in the administration of a cannabinoid to a user via inhalation for the treatment of a disease, disorder or symptom, the composition comprises an aqueous solution comprising at least one cannabinoid compound, wherein the aqueous solution has a pH of at least 9.

According to some embodiments, the use is for the treatment of a disease, disorder or symptom amenable to treatment with THC.

According to some embodiments, the disease, disorder or symptom amenable to treatment with THC is selected from the group consisting of pain, impaired neurological function, inflammation, nausea, vomiting, convulsions, low appetite and glaucoma.

It is to be understood to a person skilled in the art that THCA is an organic acid, and thus is better soluble in water, when the pH is elevated. Specifically, at higher (more basic) pH organic acids are present as salts, which are typically more water soluble then their corresponding acids.

According to some embodiments, the aqueous solution has a pH of at least 8.6. According to some embodiments, the aqueous solution has a pH of at least 8.75. According to some embodiments, the aqueous solution has a pH of at least 9. According to some embodiments, the aqueous solution has a pH of at least 9.5. According to some embodiments, the aqueous solution has a pH of at least 10. According to some embodiments, the aqueous solution has a pH of at least 10.5. According to some embodiments, aqueous solution has a pH in the range of 9.5 to 11.5. According to some embodiments, aqueous solution has a pH in the range of 9 to 11. According to some embodiments, aqueous solution has a pH in the range of 10 to 11. According to some embodiments, aqueous solution has a pH in the range of 10.5 to 11.5.

According to some embodiments, the concentration of the at least one cannabinoid compound in the aqueous solution is in the range of 0.1% to 10% w/w. According to some embodiments, the concentration of the at least one cannabinoid compound in the aqueous solution is in the range of 0.5% to 10% w/w. According to some embodiments, the concentration of the at least one cannabinoid compound in the aqueous solution is in the range of 1% to 10% w/w. According to some embodiments, the concentration of the at least one cannabinoid compound in the aqueous solution is in the range of 2% to 10% w/w. According to some embodiments, the concentration of the at least one cannabinoid compound in the aqueous solution is in the range of 4% to 6% w/w.

According to some embodiments, the percentage of the at least one cannabinoid compound in the cannabinoid compositions is within the range of 0.1 to 15% w/w. According to some embodiments, the percentage of the at least one cannabinoid compound in the cannabinoid compositions is within the range of 0.5 to 12% w/w. According to some embodiments, the percentage of the at least one cannabinoid compound in the cannabinoid compositions is within the range of 1 to 10% w/w. According to some embodiments, the percentage of the at least one cannabinoid compound in the cannabinoid compositions is within the range of 2 to 8% w/w. According to some embodiments, the percentage of the at least one cannabinoid compound in the cannabinoid compositions is within the range of 2.5 to 7.5% w/w. According to some embodiments, the percentage of the at least one cannabinoid compound in the cannabinoid compositions is within the range of 3 to 7% w/w. According to some embodiments, the percentage of the at least one cannabinoid compound in the cannabinoid compositions is within the range of 3.5 to 6.5% w/w. According to some embodiments, the percentage of the at least one cannabinoid compound in the cannabinoid compositions is within the range of 4 to 6% w/w. According to some embodiments, the percentage of the at least one cannabinoid compound in the cannabinoid compositions is within the range of 4.5 to 5.5% w/w. According to some embodiments, the percentage of the at least one cannabinoid compound in the cannabinoid compositions is about 5% w/w.

As used herein, the term “about” refers to a range of values±20%, or ±10% of a specified value. For example, the phrase “the percentage is about 5% w/w” includes ±20% of 5, or from 4% to 6%, or from 4.5% to 5.5%.

As used herein, when relating to cannabinoid percentages in liquid compositions, unless specified otherwise, the volume ratio, or w/w % is referred. For example, the phrase “the percentage of the at least one cannabinoid is within the range of 4 to 6%” refers to a liquid solution, in which a single weight unit of the solution includes from 0.04 to 0.06 the weight unit of cannabinoid. Specifically, adding 5 gr of THCA to 95 gr of water will result in a 100 gr solution of 5% THCA.

According to some embodiments, the concentration of the at least one cannabinoid in the cannabinoid composition is within the range of 0.5 to 200 mg/ml. According to some embodiments, the concentration of the at least one cannabinoid in the cannabinoid composition is within the range of 1 to 150 mg/ml. According to some embodiments, the concentration of the at least one cannabinoid in the cannabinoid composition is within the range of 2.5 to 100 mg/ml. According to some embodiments, the concentration of the at least one cannabinoid in the cannabinoid composition is within the range of 10 to 100 mg/ml. According to some embodiments, the concentration of the at least one cannabinoid in the cannabinoid composition is within the range of 20 to 90 mg/ml According to some embodiments, the concentration of the at least one cannabinoid in the cannabinoid composition is within the range of 45 to 55 mg/ml.

According to some embodiments, the at least one cannabinoid is the sole active ingredient in the cannabinoid composition. According to some embodiments, the composition comprises cannabinoid(s) as the only active ingredient.

The term “active ingredient” refers to an agent, active ingredient compound or other substance, or compositions and mixture thereof that provide some pharmacological and/or biological, often beneficial, effect.

According to some embodiments, the cannabinoid composition is a pharmaceutical composition.

According to some embodiments, the cannabinoid composition may comprise one or more active agents, other than cannabinoid(s). According to some embodiments, the one or more active agents include one or more pharmaceutically active agents. According to some embodiments, the one or more active agents are suitable or may be adjusted for inhalation. According to some embodiments, the one or more pharmaceutically active agents are directed for treatment of a medical condition through inhalation.

According to some embodiments, the medical condition is amenable to treatment with THC.

According to some embodiments, the medical condition is selected from the group consisting of pain, impaired neurological function, inflammation, nausea, vomiting, convulsions, low appetite and glaucoma.

According to some embodiments, the cannabinoid composition further comprises at least one carrier acceptable for inhalation. According to some embodiments, the carrier is stable under basic pH conditions. According to some embodiments, the carrier is water soluble under basic pH conditions. According to some embodiments, the pharmaceutical composition further comprises at least one pharmaceutically acceptable carrier, which is acceptable for inhalation. According to some embodiments, the pharmaceutically acceptable carrier is stable under basic pH conditions. According to some embodiments, the pharmaceutically acceptable carrier is water soluble under basic pH conditions.

According to some embodiments, the cannabinoid composition further comprises at least one stabilizer. According to some embodiments, the stabilizer is stable under basic pH conditions. According to some embodiments, the stabilizer is water soluble under basic pH conditions.

According to some embodiments, the cannabinoid composition further comprises at least one additive selected from the group consisting of a propellant, an anti-coughing agent and a flavorant. According to some embodiments, the cannabinoid composition further comprises at least one additive selected from the group consisting of, an anti-coughing agent and a flavorant. According to some embodiments, the cannabinoid composition further comprises at least one anti-coughing agent. According to some embodiments, the cannabinoid composition further comprises at least one flavorant.

According to some embodiments, the cannabinoid composition further comprises at least one additive at a concentration of 0.1-1% w/w. According to some embodiments, the cannabinoid composition further comprises at least one additive at a concentration of 0.1-0.5% w/w. According to some embodiments, the cannabinoid composition further comprises at least one additive at a concentration of 0.1-0.3% w/w.

According to some embodiments, the additive is approved for use in inhaling solutions. According to some embodiments, the additive is stable at basic aqueous conditions. According to some embodiments, the additive is soluble at basic aqueous conditions.

According to some embodiments, the flavorant is a sweetener. According to some embodiments, the sweetener is selected from the group of artificial sweeteners including saccharine, aspartame, dextrose and fructose.

According to some embodiments, the additive is selected from menthol, eucalyptol, tyloxapol and a combination thereof. According to some embodiments, the additive is selected from menthol, eucalyptol, tyloxapol and a combination thereof, and is present at a concentration of 0.1-0.5% w/w based on the total weight of the cannabinoid composition.

According to some embodiments, the cannabinoid composition further comprises at least one preservative. According to some embodiments, the preservative is selected from the group consisting of benzyl alcohol, propylparaben, methylparaben, benzalkonium chloride, phenylethyl alcohol, chlorobutanol, potassium sorbate, phenol, m-cresol, o-cresol, p-cresol, chlorocresol and combinations thereof.

The term “anti-coughing agent” as used herein refers to an active agent used for the suppression, alleviation or prevention of coughing and irritations and other inconveniencies in the large breathing passages that can, or may, generate coughing. Anti-coughing agent include, but are not limited to antitussives, which are used for which suppress coughing, and expectorants, which alleviate coughing, while enhancing the production of mucus and phlegm. Anti-coughing agents may ease the administration of inhaled aerosols.

According to some embodiments, the at least one anti-coughing agent is selected from expectorants, antitussives or both. According to some embodiments, the at least one anti-coughing agent is selected from the group consisting of menthol, dextromethorphan, dextromethorphan hydrobromide, hydrocodone, caramiphen dextrorphan, 3-methoxymorphinan or morphinan-3-ol, carbetapentane, codeine, acetylcysteine and combinations thereof.

As exemplified herein (e.g. Example 5) the composition of the invention provide an effective dose of THC, which is comparable to the amount of THC delivered through the lungs, by smoking cannabis directly. Without wishing to be bound by any theory or mechanism of action, the high dosage of THC that reaches the lungs by inhaling the cannabinoid composition using an electronic vaporizer is attributed to the small aerosol droplets, having MMAD within the range of about 0.2 to 4 microns. It is noted that such small droplets were maintained even at aerosol produced with high THC concentrations, of about 5%. Thus, high THC concentrations can be inhaled and reach the lungs using an electronic vaporizer adapted for the aerosolization of aqueous solutions and the cannabinoid compositions disclosed herein.

According to some embodiments, the at least one cannabinoid compound is extracted from a plant material, wherein the plant is of cannabis genus. According to some embodiments, the plant material is selected from Cannabis Indica, Cannabis Sativa and cannabis species engineered to have high THC/THCA content. According to some embodiments, the cannabis species is a THCA enriched cannabis species.

According to some embodiments, the at least one cannabinoid compound comprises tetrahydrocannabinol (THC), tetrahydrocannabinolic acid (THCA), cannabidiol (CBD), cannabidiolic acid (CBDA), salts thereof or a combination thereof. According to some embodiments, the at least one cannabinoid compound comprises THCA or a salt thereof.

According to some embodiments, the at least one cannabinoid compound comprises THCA-salt. According to some embodiments, the at least one cannabinoid compound comprises THCA-sodium salt.

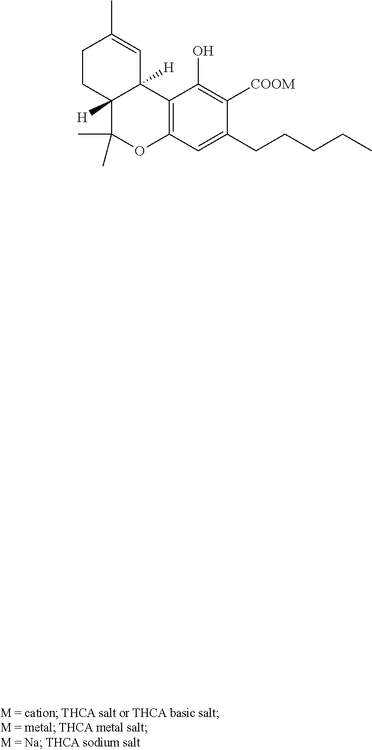

According to some embodiments, the cannabinoid composition is substantially devoid of organic solvents.

As used herein, “substantially devoid” means that a preparation or composition according to the invention that generally contains less than 3% of the stated substance, such as less than 1% or less than 0.5%.

According to some embodiments, the cannabinoid composition less than 10% w/w organic solvents. According to some embodiments, the cannabinoid composition less than 8% w/w organic solvents. According to some embodiments, the cannabinoid composition less than 6% w/w organic solvents. According to some embodiments, the cannabinoid composition less than 5% w/w organic solvents. According to some embodiments, the cannabinoid composition less than 4% w/w organic solvents. According to some embodiments, the cannabinoid composition less than 3% w/w organic solvents. According to some embodiments, the cannabinoid composition less than 2% w/w organic solvents. According to some embodiments, the cannabinoid composition less than 1% w/w organic solvents. According to some embodiments, the cannabinoid composition less than 0.5% w/w organic solvents. According to some embodiments, the cannabinoid composition less than 0.4% w/w organic solvents. According to some embodiments, the cannabinoid composition less than 0.3% w/w organic solvents. According to some embodiments, the cannabinoid composition less than 0.2% w/w organic solvents. According to some embodiments, the cannabinoid composition less than 0.1% w/w organic solvents.

According to some embodiments, the organic solvent is selected from the group consisting of: dichloromethane, chloroform. Benzene, toluene, chlorobenzene, hexane, pentane, hexanes, heptane, dimethylformamide, dimethyl sulfoxide, acetone, methanol and combinations thereof.

The heated liquid in conventional electronic vaporizers (e-cigarettes, vaping devices and the like) usually includes a composition or mixture of cannabinoids with humectants, having relatively low latent heat of vaporization, such as propylene glycol (PG) or vegetable glycerin (VG). Said composition is typically referred to as “e-juice”. The liquid mixture is typically drawn into a wicking material that is in contact with a heating element, which may consist a coil of a conducting material to be heated when electric current is driven there through. When not contacted with a liquid, or after the liquid is substantially evaporated the temperature of the coil can reach in some instances a temperature of over 800 degrees Celsius. It is to be understood that in contrast with known electronic vaporizer compositions, which employ THC formulations comprising PG and/or VG, which are hazardous upon heating and decomposition, the present invention provides aqueous cannabinoid formulations and do not require hazardous organic solvents.

According to some embodiments, the cannabinoid composition is in liquid form. According to some embodiments, the cannabinoid composition comprises at least 40% w/w water. According to some embodiments, the cannabinoid composition comprises at least 50% w/w water. According to some embodiments, the cannabinoid composition comprises at least 60% w/w water. According to some embodiments, the cannabinoid composition comprises at least 70% w/w water. According to some embodiments, the cannabinoid composition comprises at least 75% w/w water. According to some embodiments, the cannabinoid composition comprises at least 80% w/w water. According to some embodiments, the cannabinoid composition comprises at least 85% w/w water. According to some embodiments, the cannabinoid composition comprises at least 90% w/w water. It is to be understood that the phrase “cannabinoid composition comprises at least 90% w/w water” means that each gram of the total composition includes at least 900 milligrams of water and at most 100 milligrams of materials other than water. According to some embodiments, the cannabinoid composition comprises more than 90% w/w water.

According to some embodiments, there is provided a process for preparing a cannabinoid composition. According to some embodiments, there is provided a process for preparing the cannabinoid composition disclosed herein.

According to some embodiments, the aqueous solution is prepared by a process comprising the steps of:
(a) contacting cannabis plant material with an aqueous base, to form an aqueous solution comprising the at least one cannabinoid, and a water insoluble plant material; and
(b) separating the aqueous solution comprising the at least one cannabinoid from the insoluble plant material.

According to some embodiments, the aqueous solution is prepared by a process consisting essentially the steps of:
(a) contacting cannabis plant material with an aqueous base, to form an aqueous solution comprising the at least one cannabinoid, and a water insoluble plant material; and
(b) separating the aqueous solution comprising the at least one cannabinoid from the insoluble plant material.

The term aqueous base refers to any solution, emulsion or suspension comprising at least 50% water and having a pH above 8.

According to some embodiments, the aqueous base comprises sodium hydroxide, potassium hydroxide, lithium hydroxide, magnesium hydroxide, sodium carbonate, potassium carbonate or a combination thereof. Each possibility represents a separate embodiment. According to some embodiments, the aqueous base is selected from the group consisting of aqueous sodium hydroxide, aqueous potassium hydroxide, aqueous lithium hydroxide, aqueous magnesium hydroxide, aqueous sodium carbonate and aqueous potassium. According to some embodiments, the aqueous base comprises sodium hydroxide.

According to some embodiments, the aqueous base is aqueous sodium hydroxide. According to some embodiments, the aqueous base is aqueous sodium hydroxide at a concentration in the range of 0.005M to 1M.

According to some embodiments, the aqueous base comprises a hydroxide anion at a concentration in the range of 0.001M to 0.5M. According to some embodiments, the aqueous base comprises a hydroxide anion at a concentration in the range of 0.05M to 0.5M. According to some embodiments, the aqueous base comprises a hydroxide anion at a concentration in the range of 0.1M to 0.25M

According to some embodiments, the process further comprises a step of grinding the cannabis plant material prior to step (a).

According to some embodiments, the contacting of step (a) is maintained for at least 1 hour. According to some embodiments, the contacting of step (a) is maintained for at least 2 hours. According to some embodiments, the contacting of step (a) is maintained for at least 4 hours. According to some embodiments, the contacting of step (a) is maintained for at least 6 hours. According to some embodiments, the contacting of step (a) is maintained for at least 8 hours. According to some embodiments, the contacting of step (a) is maintained for at least 10 hours. According to some embodiments, the contacting of step (a) is maintained for at least 12 hours. According to some embodiments, the contacting of step (a) is maintained for at least 15 hours. According to some embodiments, the contacting of step (a) is maintained for at least 18 hours. According to some embodiments, the contacting of step (a) is maintained for at least 21 hours. According to some embodiments, the contacting of step (a) is maintained for at least 24 hours.

According to some embodiments, the separation of step (b) is performed by centrifugation.

According to some embodiments, step (a) further comprises applying pressure on the cannabis plant material in the aqueous base. According to some embodiments, step (a) further comprises macerating the cannabis plant material in the aqueous base.

According to some embodiments, the cannabis plant material of step (a) comprises tetrahydrocannabinolic acid (THCA). According to some embodiments, the cannabis plant material of step (a) comprises a THCA-enriched cannabis species.

According to some embodiments, the process is devoid of steps of extraction with an organic solvent.

According to some embodiments, the process further comprises the steps of:
(c) adding an acid to the aqueous solution comprising the at least one cannabinoid to a pH in the range of 1-5, thereby precipitating the at least one cannabinoid and forming an acidic aqueous solution;
(d) separating the precipitated at least one cannabinoid from the acidic aqueous solution; and
(e) dissolving the precipitated at least one cannabinoid in a second aqueous base, thereby forming a purified aqueous solution comprising the at least one cannabinoid.

According to some embodiments, the acid is a mineral acid.

According to some embodiments, the pH of the acidic aqueous solution of step (c) is in the range of 2 to 5.5. According to some embodiments, the pH of the acidic aqueous solution of step (c) is in the range of 2.5 to 5.5. According to some embodiments, the pH of the acidic aqueous solution of step (c) is in the range of 3 to 5. According to some embodiments, the pH of the acidic aqueous solution of step (c) is in the range of 3.5 to 4.5.

According to some embodiments, the separation of step (d) is performed by centrifugation.

According to some embodiments, the second aqueous base of step (e) comprises a hydroxide anion at a concentration in the range of 0.001M to 0.5M. According to some embodiments, the second aqueous base of step (e) comprises a hydroxide anion at a concentration in the range of 0.002M to 0.5M. According to some embodiments, the second aqueous base of step (e) comprises a hydroxide anion at a concentration in the range of 0.005M to 0.5M. According to some embodiments, the second aqueous base of step (e) comprises a hydroxide anion at a concentration in the range of 0.01M to 0.5M.

According to some embodiments, there is provided a method of delivering a cannabinoid to a user of an electronic vaporizer via inhalation, the method comprising the steps of:
(i) providing the cannabinoid composition as disclosed herein; and
(ii) aerosolizing the cannabinoid composition of step (a) with an electronic vaporizer, to form an inhalable aerosol.

According to some embodiments, there is provided a method of delivering a cannabinoid to a user of an electronic vaporizer via inhalation, the method comprising the steps of:
(i) providing a cannabinoid composition comprising an aqueous solution comprising at least one cannabinoid compound, wherein the aqueous solution has a pH of at least 8.5; and
(ii) aerosolizing the cannabinoid composition of step (a) with an electronic vaporizer, to form an inhalable aerosol.

According to some embodiments, there is provided a treating a medical condition amenable to treatment with THC, the method comprising the steps of:
(i) providing a cannabinoid composition as disclosed herein;
(ii) aerosolizing the cannabinoid composition of step (a) with an aerosol generating device; and
(iii) delivering the inhalable aerosol of step (ii) to a subject in need thereof, thereby treating the medical condition amenable to treatment with THC.

According to some embodiments, there is provided a method of treating a medical condition amenable to treatment with THC, the method comprising the steps of:
(i) providing a cannabinoid composition comprising an aqueous solution comprising at least one cannabinoid compound, wherein the aqueous solution has a pH of at least 8.5;
(ii) aerosolizing the cannabinoid composition of step (a) with an aerosol generating device; and
(iii) delivering the inhalable aerosol of step (ii) to a subject in need thereof, thereby treating the medical condition amenable to treatment with THC.

According to some embodiments, the aerosol generating device is selected from the group consisting of an electronic vaporizer, a nebulizer and an inhaler. According to some embodiments, the aerosol generating device is an electronic vaporizer. According to some embodiments, the aerosol generating device is a nebulizer. According to some embodiments, the aerosol generating device is an inhaler. According to some embodiments, the aerosol generating device is an inhaler or a nebulizer.

According to some embodiments, the electronic vaporizer is a vaping device or an electronic cigarette. According to some embodiments, the electronic vaporizer is a vaping device. According to some embodiments, the electronic vaporizer is an electronic cigarette.

The term “aerosol generating device” refer to a device configured to produce a vapor or aerosol from a liquid or solid composition. aerosol generating devices are typically used to deliver a solid or liquid (including semi liquid) composition to a subject in need thereof in a inhalable form (i.e. in a substantially gaseous form). Aerosol generating devices include nebulizers and inhalers, which typically produce aerosols by application of mechanical force on the compositions (e.g. by gas flow or vacuum), and to vaporizers and electronic cigarettes, which typically heating unit(s) and produce aerosols by vaporizing the composition. In both instances, the composition is delivered through an outlet, wherein in the latter instances (i.e. vaporizers and electronic cigarettes) the vapor is usually at least partially being condensed to form droplets of the composition, through the delivery.

The present composition is typically aerosolized by aerosol generating devices, which include heating units, and are typically conventionally referred to as vaporizers. Specifically, the typical convention is that aerosol generating devices for aerosolizing nicotine/tobacco compositions are called electronic cigarettes, whereas devices for aerosolizing cannabinoid/cannabis compositions are called vaporizer or vaping devices.

As used herein the term “electronic vaporizer” refers either to electronic cigarettes or to vaporizer and vaping devices.

According to some embodiments, the medical condition is selected from the group consisting of pain, impaired neurological function, inflammation, nausea, vomiting, convulsions, low appetite and glaucoma.

According to some embodiments, the inhalable aerosol is inhaled by the user of the electronic vaporizer. According to some embodiments, the inhalable aerosol is inhaled by the subject. According to some embodiments, the method further comprises the step of inhaling the inhalable aerosol by a user. According to some embodiments, the method further comprises the step of inhaling the inhalable aerosol by the subject. According to some embodiments, the method further comprises the step of inhaling the inhalable aerosol by a user of an electronic vaporizer.

According to some embodiments, the delivering of the cannabinoid to a user comprises delivering of the cannabinoid to a user comprises delivering the cannabinoid to the respiratory system of the user. According to some embodiments, the delivering of the cannabinoid to the subject comprises delivering of the cannabinoid to a user comprises delivering the cannabinoid to the respiratory system of the subject.

It is to be understood that the embodiment related to cannabinoid composition and/or the pharmaceutical composition above, may apply for any of the methods disclosed herein. Specifically, wherein the method is for treatment of a subject, the cannabinoid composition may be a pharmaceutical composition, according to some embodiments.

According to some embodiments, inhalable aerosol formed in step (ii) has a pH in the range of 5.5 to 7.5.

As elaborated above, the pH of the cannabinoid composition is highly basic, whereas the pH of the aerosol produced therefrom is typically substantially neutral, according to some embodiments. This may be the result of the formation of the neutral compound THC from THCA basic salt. As detailed in Examples 1 and 2, the aerosol produced by the aerosolization (or a number of aerosolization events) of the cannabinoid composition may be collected, and its pH measured conveniently.

According to some embodiments, the aerosolizing of step (ii) comprises heating the cannabinoid composition of step (i) with the electronic vaporizer. According to some embodiments, the aerosolizing of step (ii) comprises heating the cannabinoid composition of step (i) with the aerosol generating device.

According to some embodiments, the aqueous solution of step (i) has a pH in the range of 10.5 to 11.5. According to some embodiments, the concentration of the at least one cannabinoid compound in the aqueous solution of step (i) is in the range of 4% to 6% w/w. According to some embodiments, the at least one cannabinoid compound is extracted from a plant material, wherein the plant is of cannabis genus. According to some embodiments, the at least one cannabinoid compound comprises tetrahydrocannabinol (THC), tetrahydrocannabinolic acid (THCA), cannabidiol (CBD), cannabidiolic acid (CBDA), salts thereof or a combination thereof. According to some embodiments, the at least one cannabinoid compound comprises THCA or a salt thereof.

According to some embodiments, the cannabinoid composition of step (i) is substantially devoid of organic solvents. According to some embodiments, the cannabinoid composition of step (i) is in liquid form and comprising at least 90% w/w water.

According to some embodiments, the aqueous solution of step (i) is prepared the process disclosed herein. Specifically, according to some embodiments, the aqueous solution of step (i) is prepared by a process comprising the steps of: (a) contacting cannabis plant material with an aqueous base, to form an aqueous solution comprising the at least one cannabinoid, and a water insoluble plant material; and (b) separating the aqueous solution comprising the at least one cannabinoid from the insoluble plant material.

According to some embodiments, step (a) further comprises macerating the cannabis plant material in the aqueous base. According to some embodiments, the process of preparing aqueous solution of step (i) is devoid of steps of extraction with an organic solvent. According to some embodiments, the process of preparing aqueous solution of step (i) further comprises the steps of: adding an acid to the aqueous solution comprising the at least one cannabinoid to a pH in the range of 1-5, thereby precipitating the at least one cannabinoid and forming an acidic aqueous solution; (d) separating the precipitated at least one cannabinoid from the acidic aqueous solution; and (e) dissolving the precipitated at least one cannabinoid in a second aqueous base, thereby forming a purified aqueous solution comprising the at least one cannabinoid.

According to some embodiments, step (ii) of the method of delivering a cannabinoid to a user of an electronic vaporizer via inhalation, comprises: providing an electronic vaporizer configured for the aerosolization of aqueous solutions, and aerosolizing the cannabinoid composition of step (a) with the electronic vaporizer, to form the inhalable aerosol.

According to some embodiments, step (ii) of the method of treating a medical condition amenable to treatment with THC, comprises: providing an aerosol generating device configured for the aerosolization of aqueous solutions, and aerosolizing the cannabinoid composition of step (a) with the aerosol generating device, to form the inhalable aerosol.

As detailed above, the methods of the current invention are effective in delivering THC to the respiratory system of the electronic vaporizer user and/or to the respiratory system of the subject in need of treatment with said cannabinoid, according to some embodiments.

As used herein, “respiratory system” refers to the system of organs in the body responsible for the intake of oxygen and the expiration of carbon dioxide. The system generally includes all the air passages from the nose to the pulmonary alveoli. In mammals it is generally considered to include the lungs, bronchi, bronchioles, trachea, nasal passages, and diaphragm. For purposes of the present disclosure, delivery of a drug to the “respiratory system” indicates that a drug is delivered to one or more of the air passages of the respiratory system, in particular to the lungs.

The correlation between droplet size and deposition thereof in the respiratory tract has been established. Droplets around 10 micron in diameter are suitable for deposition in the oropharynx and the nasal area; droplets below around 4 micron in diameter are suitable for deposition in the central airways and may be especially beneficial for delivery of cannabinoid the subjects in a need thereof. The droplets formed by aerosolizing the cannabinoid composition of the current invention are small, having droplet size in the range of 0.1 to 5 micron, according to some embodiments.

According to some embodiments, there is provided an electronic vaporizer cartridge comprising a liquid container, wherein the liquid container contains a cannabinoid as disclosed herein. According to some embodiments, there is provided an electronic vaporizer cartridge comprising a liquid container, wherein the liquid container contains a cannabinoid composition comprising an aqueous solution comprising at least one cannabinoid compound, wherein the aqueous solution has a pH of at least 8.5. According to some embodiments, there is provided an vaporizer cigarette comprising a liquid container, wherein the liquid container contains a cannabinoid composition as disclosed herein. According to some embodiments, there is provided an electronic vaporizer comprising a liquid container, wherein the liquid container contains a cannabinoid composition comprising an aqueous solution comprising at least one cannabinoid compound, wherein the aqueous solution has a pH of at least 8.5.

According to some embodiments, there is provided an aerosol generating device cartridge comprising a liquid container, wherein the liquid container contains a cannabinoid as disclosed herein. According to some embodiments, there is provided an aerosol generating device cartridge comprising a liquid container, wherein the liquid container contains a cannabinoid composition comprising an aqueous solution comprising at least one cannabinoid compound, wherein the aqueous solution has a pH of at least 8.5. According to some embodiments, there is provided an aerosol generating device comprising a liquid container, wherein the liquid container contains a cannabinoid as disclosed herein. According to some embodiments, there is provided an aerosol generating device comprising a liquid container, wherein the liquid container contains a cannabinoid composition comprising an aqueous solution comprising at least one cannabinoid compound, wherein the aqueous solution has a pH of at least 8.5.

It is to be understood that embodiments referring to the cannabinoid compositions presented herein also apply to the cannabinoid compositions contained in any one of the electronic vaporizer, electronic vaporizer cartridge, aerosol generating device and/or aerosol generating device cartridge disclosed herein. Specifically, an electronic vaporizer is an aerosol generating device, which comprises a heating unit assisting in the production of the aerosol.

According to some embodiments, the aerosol generating device comprises a liquid absorbing material configured to absorb liquids. According to some embodiments, the electronic vaporizer comprises a liquid absorbing material configured to absorb liquids.

According to some embodiments, the cannabinoid composition is absorbed within the liquid absorbing material. According to some embodiments, the cannabinoid composition is partially absorbed within the liquid absorbing material. According to some embodiments, the liquid absorbing material is in contact with the liquid container. According to some embodiments, the liquid absorbing material is selected from a wick and a sponge. According to some embodiments, the liquid absorbing material is a wick. According to some embodiments, the liquid absorbing material is a sponge.

The term ‘liquid absorbing material’ as used herein refer to any material that is capable of incorporating, taking in, drawing in or soaking liquids, and upon applying physical pressure thereto, release a portion or the entire amount/volume of the absorbed liquid. The physical pressure may be achieved for example by pressing the material against a solid structure.

According to some embodiments, the aqueous solution has a pH in the range of 10.5 to 11.5. According to some embodiments, concentration of the at least one cannabinoid compound in the aqueous solution is in the range of 4% to 6% w/w. According to some embodiments, the at least one cannabinoid compound comprises THCA or a salt thereof. According to some embodiments, the cannabinoid composition is substantially devoid of organic solvents. According to some embodiments, the cannabinoid composition is in liquid form and comprises at least 90% w/w water. According to some embodiments, the aqueous solution is prepared by a process comprising the steps of: (a) contacting cannabis plant material with an aqueous base, to form an aqueous solution comprising the at least one cannabinoid, and a water insoluble plant material; and (b) separating the aqueous solution comprising the at least one cannabinoid from the insoluble plant material.

According to some embodiments, there is provided an aerosol composition comprising tetrahydrocannabinol (THC) and tetrahydrocannabinolic acid (THCA) at a total weight of 1-8% w/w based on the total weight of the aerosol composition, and water 70-99% w/w based on the total weight of the aerosol composition, wherein the aerosol comprising droplets having an mass median aerodynamic diameter (MMAD) of at most 5 microns.

As used herein the term “aerosol” refers to a suspension of solid or liquid particles in a gas. As used herein “aerosol” may be used generally to refer to a drug that has been vaporized, nebulized, or otherwise converted from a solid or liquid form to an inhalable form including suspended solid or liquid drug particles. According to some embodiments, the drug particles include THC particles.

According to some embodiments, the aerosol composition further comprises tetrahydrocannabinolic acid (THCA).

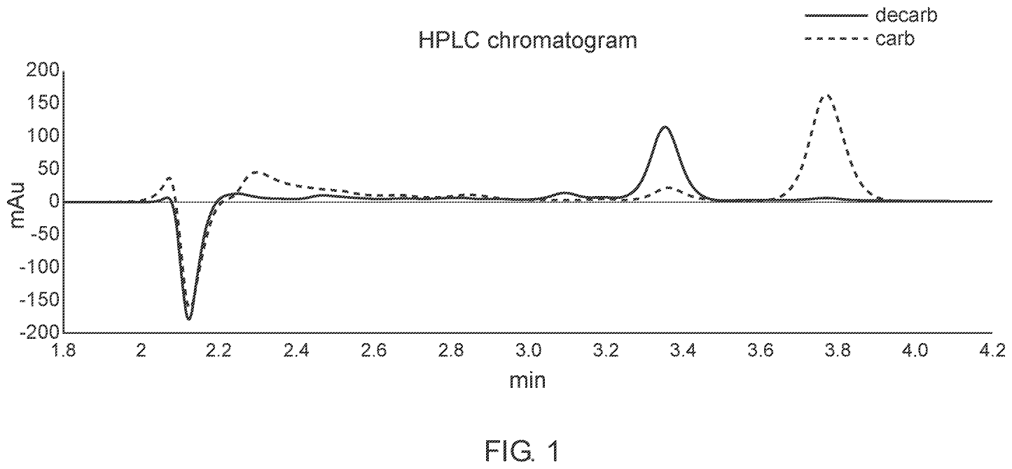

Specifically, as shown in Example 4, the cannabinoid composition of the current invention comprises THCA as a main component, and upon aerosolization, it undergoes decarboxylation, to form an aerosol comprising mainly THC. However, according to some embodiments, traces of THCA may still be present in the aerosol, as shown in FIG. 1.

According to some embodiments, the aerosol composition is having a pH in the range of 5.5 to 7.5. Specifically, upon collection of the aerosol, the pH was measured to be substantially neutral, indicating the substantial disappearance of THCA and formation of the pH neutral THC.

According to some embodiments, the aerosol composition comprises droplets having a mass median aerodynamic diameter (MMAD) of at most 5 microns. According to some embodiments, the aerosol composition comprises droplets having a mass median aerodynamic diameter (MMAD) of at most 4 microns. According to some embodiments, the aerosol composition comprises droplets having a mass median aerodynamic diameter (MMAD) of at most 3 microns. According to some embodiments, the aerosol composition comprises droplets having a mass median aerodynamic diameter (MMAD) of at most 2 microns. According to some embodiments, the aerosol composition comprises droplets having a mass median aerodynamic diameter (MMAD) of at most 1 microns. According to some embodiments, the aerosol composition comprises droplets having a mass median aerodynamic diameter (MMAD) of at most 0.8 microns. According to some embodiments, the aerosol composition comprises droplets having a mass median aerodynamic diameter (MMAD) of at most 0.6 microns. According to some embodiments, the aerosol composition comprises droplets having a mass median aerodynamic diameter (MMAD) of at most 0.5 microns.

It was surprisingly found that aerosolization of a formulation as disclosed herein, results in droplets having a mass median aerodynamic diameter (MMAD) sufficiently small so as to reach the lungs, rather than precipitate on their way thereto. The small droplets reaching the lungs enable efficient respiratory delivery of the cannabinoid(s). This is an overall advantage as maximizing the delivery of cannabinoid(s) to the lungs, while minimizing its deposition in the mouth and throat are considered highly beneficial.

The terms ‘droplet size’ and ‘mass median aerodynamic diameter’, also known as MMAD, as used herein are interchangeable. MMAD is commonly considered as the median particle diameter by mass. MMAD may be evaluated by plotting droplet size vs. the cumulative mass fraction (%) in the aerosol. MMAD may then be determined according to the interpolated droplet size corresponding to the point, where the cumulative mass fraction is 50%. This point represents the estimated values of particle sizes, above which the droplets are responsible to half to masses and below which the droplets are responsible to the other halves, in each solution.

According to some embodiments, the aerosol comprises droplets having a Geometric Standard Diameter (GSD) within the range of about 0.2-7 micron. According to some embodiments, the aerosol comprises droplets having a GSD within the range of about 0.2-5 micron.

According to some embodiments, the aerosol composition further comprises an additive selected from the group consisting of a carrier, a preservative, an anti-coughing agent, a propellant and a flavorant.

According to some embodiments, the aerosol composition further comprises at least one carrier acceptable for inhalation. According to some embodiments, the carrier is stable under basic pH conditions. According to some embodiments, the carrier is water soluble under basic pH conditions. According to some embodiments, the aerosol composition further comprises at least one pharmaceutically acceptable carrier, which is acceptable for inhalation. According to some embodiments, the pharmaceutically acceptable carrier is stable under basic pH conditions. According to some embodiments, the pharmaceutically acceptable carrier is water soluble under basic pH conditions. According to some embodiments, the aerosol composition further comprises at least one stabilizer. According to some embodiments, the stabilizer is stable under basic pH conditions. According to some embodiments, the stabilizer is water soluble under basic pH conditions. According to some embodiments, the aerosol composition further comprises at least one additive selected from the group consisting of a propellant, an anti-coughing agent and a flavorant. According to some embodiments, the aerosol composition further comprises at least one additive selected from the group consisting of, an anti-coughing agent and a flavorant. According to some embodiments, the aerosol composition further comprises at least one anti-coughing agent. According to some embodiments, the aerosol composition further comprises at least one flavorant. According to some embodiments, the aerosol composition further comprises at least one additive at a concentration of 0.1-1% w/w. According to some embodiments, the aerosol composition further comprises at least one additive at a concentration of 0.1-0.5% w/w. According to some embodiments, the aerosol composition further comprises at least one additive at a concentration of 0.1-0.3% w/w. According to some embodiments, the additive is approved for use in inhaling solutions. According to some embodiments, the additive is stable at basic aqueous conditions. According to some embodiments, the additive is soluble at basic aqueous conditions. According to some embodiments, the flavorant is a sweetener. According to some embodiments, the sweetener is selected from the group of artificial sweeteners including saccharine, aspartame, dextrose and fructose. According to some embodiments, the aerosol composition further comprises at least one preservative. According to some embodiments, the preservative is selected from the group consisting of benzyl alcohol, propylparaben, methylparaben, benzalkonium chloride, phenylethyl alcohol, chlorobutanol, potassium sorbate, phenol, m-cresol, o-cresol, p-cresol, chlorocresol and combinations thereof. According to some embodiments, the at least one anti-coughing agent is selected from expectorants, antitussives or both. According to some embodiments, the at least one anti-coughing agent is selected from the group consisting of menthol, dextromethorphan, dextromethorphan hydrobromide, hydrocodone, caramiphen dextrorphan, 3-methoxymorphinan or morphinan-3-ol, carbetapentane, codeine, acetylcysteine and combinations thereof.

According to some embodiments, the additive is selected from menthol, eucalyptol, tyloxapol and a combination thereof. According to some embodiments, the additive is selected from menthol, eucalyptol, tyloxapol and a combination thereof, and is present at a concentration of 0.1-0.5% w/w based on the total weight of the cannabinoid composition.

According to some embodiments, the aerosol composition is prepared by aerosolizing a cannabinoid composition as disclosed herein. According to some embodiments, the aerosol composition is prepared by aerosolizing a cannabinoid composition comprising an aqueous solution comprising THCA, wherein the aqueous solution has a pH of at least 9.

### EXAMPLES

### Example 1: Preparation of Formulation for Inhalation

The formulation for inhalation analyzed in the experiments below included a clear basic aqueous solution of tetrahydrocannabinolic acid (THCA) adjusted to pH˜11. The solution was prepared by grinding a 1000 gr sample of THCA-enriched cannabis species and placing the ground plant material in a glass vessel. About 12 L aqueous solution of 0.1M sodium hydroxide was added to the glass vessel and the mixture was left over night. All the material was then transferred from the glass vessel to a stainless-steel mesh and the plant material was macerated by application of physical pressure. The liquid contents were then centrifuged and the supernatant was collected as clear solution. The solution was visibly clear and its pH was measured to be about 11. The solution was measured to contain about 5% w/w sodium tetrahydrocannabinolate (tetrahydrocannabinolic acid sodium salt). The solution was ready for inhalation using an electronic vaporizer.

The formulation was aerosolized from an electronic vaporizer designed by Omega Life Science for aerosolization of aqueous compositions. The aerosol was collected and its pH was measured to be substantially neutral, indicating that the THCA underwent decarboxylation to form the pH neutral compound THC in the aerosol.

### Example 2: Preparation of Formulation for Inhalation

The formulation for inhalation analyzed in the experiments below included a clear basic aqueous solution of tetrahydrocannabinolic acid (THCA) adjusted to pH˜11. The solution was prepared by grinding a 1000 gr sample of THCA-enriched cannabis species and placing the ground plant material in a glass vessel. About 12 L aqueous solution of 0.1M sodium hydroxide was added to the glass vessel and the mixture was left over night. All the material was then transferred from the glass vessel to a stainless-steel mesh and the plant material was macerated by application of physical pressure. The liquid contents were then centrifuged and the supernatant was collected as clear solution. Thereafter, the clear solution was added concentrated hydrochloric acid until the pH reached about 4. As a result, tetrahydrocannabinolic acid started to precipitate. The formed suspension was centrifuged and the solids were separated. The centrifugation and solid separation steps were repeated twice more with sequential additions of water. The solid tetrahydrocannabinolic acid was the solubilized in 0.01M aqueous sodium hydroxide. The solution was visibly clear and its pH was measured to be about 11. The solution was measured to contain about 5% w/w sodium tetrahydrocannabinolate (tetrahydrocannabinolic acid sodium salt). The solution was ready for inhalation with an electronic vaporizer.

The formulation was aerosolized from an electronic vaporizer as mentioned in Example 1. The aerosol was collected and its pH was measured to be substantially neutral, indicating that the THCA underwent decarboxylation to form the pH neutral compound THC in the aerosol.

### Example 3: Preparation of Formulation for Inhalation

The formulation for inhalation analyzed in the experiments below included a clear basic aqueous solution of tetrahydrocannabinolic acid (THCA) adjusted to pH˜11. The solution was prepared by grinding a 1000 gr sample of THCA-enriched cannabis species and placing the ground plant material in a glass vessel. About 12 L aqueous solution of 0.1M sodium hydroxide was added to the glass vessel and the mixture was left over night. All the material was then transferred from the glass vessel to a stainless-steel mesh and the plant material was macerated by application of physical pressure. The liquid contents were then centrifuged and the supernatant was collected as clear solution. Thereafter, the clear solution was added concentrated hydrochloric acid until the pH reached about 4. As a result, tetrahydrocannabinolic acid started to precipitate. The formed suspension was centrifuged and the solids were separated. The centrifugation and solid separation steps were repeated twice more with sequential additions of water. The solid tetrahydrocannabinolic acid was then solubilized in 50 mM Carbonate buffer pH 10. The solution was visibly clear and its pH was measured to be between 8-9, typically pH 8.6. The solution was measured to contain about 5% w/w sodium tetrahydrocannabinolate (tetrahydrocannabinolic acid sodium salt). The solution was ready for inhalation with an electronic cigarette.

The formulation was aerosolized from an electronic cigarette as mentioned in Example 1. The aerosol was collected and its pH was measured to be substantially neutral, indicating that the THCA underwent decarboxylation to form the pH neutral compound THC in the aerosol.

### Example 4: Analysis of the Formulation for Inhalation

The exemplary formulation solution was checked for the relative amounts of the cannabinoids THC and THCA in a Dionex ultimate 3000 HPLC system with the mobile phase being 90% acetonitrile/10% water/0.1% formic acid and the stationary phase being reverse phase C18 column. The column oven temperature was set to 35° C. and the flow was set to 1 ml/min. The UV detection was at 220 nm. The elution time were compared with elution times of THC and THCA as known in the literature.

FIG. 1 is showing two overlaying chromatograms. The dotted trend line represents the chromatogram resulting from the elution of the formulation of Example 1, without further processing, with the mobile phase being 90% acetonitrile/10% water/0.1% formic acid. This chromatogram shows a large peak at retention time of about 3.8 minutes, which is comparable with the literature value of THCA at similar elution conditions, and a small peak at about 3.35 minutes, which is comparable with the literature value of THC at similar elution conditions. Therefore, it is concluded that the formulation of the current invention comprises mainly THCA, which in basic conditions appears as a basic salt.

The second chromatogram of FIG. 1, represented by a full line, resulted from the elution in the conditions above of an aerosol collected from an electronic vaporizer as mentioned in Example 1, after aerosolizing the formulation of Example 1. This chromatogram of FIG. 1 shows a large peak at retention time of about 3.35, which is indicative of THC; and a very small peak at retention time of about 3.8, indicating THCA. Therefore, it is concluded that the aerosol formed by heating the formulation of the current invention with an electronic vaporizer comprises mainly THC, which is the active cannabinoid form. This also may explain the neutral pH of the aerosol. Without wishing to be bound by any theory or mechanism of action, upon the heating of the THCA, the majority thereof is decarboxylated to for THC. Since the heating process is rapid, some of the THCA is evaporated before decarboxylating and thus it appears in the aerosol.

### Example 5: Mass Distribution on Impactor Parts

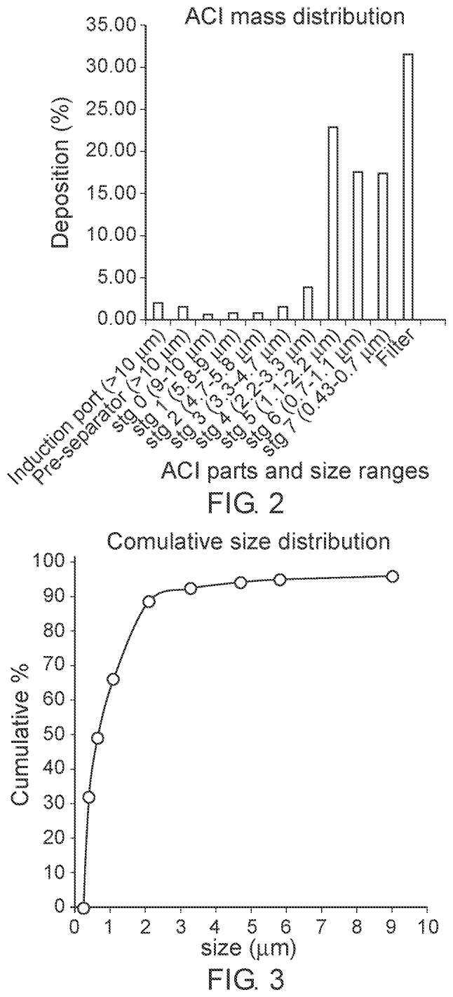

Particle size distribution testing was conducted using cascade impactor validated method with the basic aqueous solution of tetrahydrocannabinolic acid of Example 1. The limits for the median diameter range from 0.4 to 0.8 micron and the limit on the sub 5 micron particles/droplets was set at 90%. The results are presented in FIG. 2 and relate to the formulation of example 1 aerosolized with an electronic vaporizer mentioned in Example 1.

Relative mass of the aerosolized solution was measured against its particle size, which was measured between 0.43 micrometers and over 10 micrometers.

FIG. 2 is a chart representing Mass Distribution on Impactor parts in an aerosol depicting the relative mass of the aerosol in each particle diameter size group, where the particle diameter groups are 0.43 to 0.7 microns; 0.7 to 1.1 microns; 1.1 to 2.2 microns; 2.2 to 3.3 microns; 3.3 to 4.7 microns; 4.7 to 5.8 microns; 5.8 to 9 microns; and over 10 microns.

As can be seen in FIG. 2, the majority of aerosol mass was provided in droplets having diameters in the range of 0.43 to 2.2 microns.

Finally, FIG. 3 is a chart representing cumulative Mass Distribution of the aerosol in the experiment. It depicts the cumulative mass fraction vs. the droplet size in micrometers. The 50% mark in the cumulative percentage axis represents the estimated value of particle size, above which the droplets are responsible to half to mass and below which the droplets are responsible to the other half. Again, it is seen that half of the mass was delivered in droplets having diameters below about 0.8 microns.

Although the invention is described in conjunction with specific embodiments thereof, it is evident that numerous alternatives, modifications and variations that are apparent to those skilled in the art may exist. It is to be understood that the invention is not necessarily limited in its application to the details of construction and the arrangement of the components and/or methods set forth herein. Other embodiments may be practiced, and an embodiment may be carried out in various ways. Accordingly, the invention embraces all such alternatives, modifications and variations that fall within the scope of the appended claims.

1-52. (canceled)

53. A cannabinoid composition suitable for administration of a cannabinoid via inhalation, the cannabinoid composition comprises an aqueous solution comprising at least one cannabinoid acid or a salt thereof, wherein the aqueous solution has a pH of at least 8.5.

54. The cannabinoid composition of claim 53, wherein the administration of the cannabinoid acid or salt thereof via inhalation comprises generating an inhalable aerosol upon heating the at least one cannabinoid acid or salt thereof composition in an electronic vaporizer.

55. The cannabinoid composition of claim 54, wherein the inhalable aerosol has a pH in the range of 5.5 to 7.5.

56. The cannabinoid composition of claim 53, wherein the aqueous solution has a pH in the range of 10.5 to 11.5.

57. The cannabinoid composition of claim 53, wherein the concentration of the at least one cannabinoid acid or salt thereof in the aqueous solution is in the range of 4% to 6% w/w.

58. The cannabinoid composition of claim 53, wherein the at least one cannabinoid acid or salt thereof comprises tetrahydrocannabinolic acid (THCA), cannabidiolic acid (CBDA), salts thereof or a combination thereof.

59. The cannabinoid composition of claim 58, wherein the at least one cannabinoid acid or salt thereof comprises THCA or a salt thereof.

60. The cannabinoid composition of claim 53, substantially devoid of organic solvents.

61. The cannabinoid composition of claim 53, which is in liquid form and comprising at least 90% w/w water.

62. The cannabinoid composition of claim 53, wherein the aqueous solution is prepared by a process comprising the steps of:
(a) contacting cannabis plant material with an aqueous base, to form an aqueous solution comprising the at least one cannabinoid acid or salt thereof, and a water insoluble plant material; and
(b) separating the aqueous solution comprising the at least one cannabinoid acid or salt thereof from the insoluble plant material.

63. The cannabinoid composition of claim 62, wherein the aqueous base comprises sodium hydroxide, potassium hydroxide, lithium hydroxide, magnesium hydroxide, sodium carbonate, potassium carbonate or a combination thereof.

64. The cannabinoid composition of claim 63, wherein the aqueous base comprises a hydroxide anion at a concentration in the range of 0.001M to 0.5M.

65. The cannabinoid composition of claim 63, wherein the process further comprises a step of grinding the cannabis plant material prior to step (a).

66. The cannabinoid composition of claim 63, wherein step (a) further comprises macerating the cannabis plant material in the aqueous base.

67. The cannabinoid composition of claim 63, wherein the process is devoid of steps of extraction with an organic solvent.

68. The cannabinoid composition of claim 63, wherein the process further comprises the steps of:
(c) adding an acid to the aqueous solution comprising the at least one cannabinoid acid or salt thereof to a pH in the range of 1-5, thereby precipitating the at least one cannabinoid acid or salt thereof and forming an acidic aqueous solution;
(d) separating the precipitated at least one cannabinoid acid or salt thereof from the acidic aqueous solution; and
(e) dissolving the precipitated at least one cannabinoid acid or salt thereof in a second aqueous base, thereby forming a purified aqueous solution comprising the at least one cannabinoid acid or salt thereof.

69. The cannabinoid composition of claim 68, wherein the pH of the acidic aqueous solution of step (c) is in the range of 3.5 to 4.5.

70. A method of delivering a cannabinoid to a user of an electronic vaporizer via inhalation, the method comprising the steps of:
(i) providing a cannabinoid composition comprising an aqueous solution comprising at least one cannabinoid acid or salt thereof, wherein the aqueous solution has a pH of at least 8.5; and
(ii) aerosolizing the cannabinoid composition of step (a) with an electronic vaporizer, to form an inhalable aerosol,
wherein the inhalable aerosol is inhaled by the user of the electronic vaporizer.

71. The method of claim 63, wherein the aerosolizing of step (ii) comprises heating the cannabinoid composition of step (i) with the electronic vaporizer.

72. An electronic vaporizer cartridge comprising a liquid container, wherein the liquid container contains a cannabinoid composition comprising an aqueous solution comprising at least one cannabinoid acid or salt thereof, wherein the aqueous solution has a pH of at least 8.5.
